SEQUENCE LISTING (6 sequences)

SEQ ID NO: 1 - DNA, 20 bp, Synthetic construct
  source 1..20: mol_type = other DNA
tgtcactgctactgctgctg

SEQ ID NO: 2 - DNA, 20 bp, Synthetic construct
  source 1..20: mol_type = other DNA
gtgaggtggatcggttgtag

SEQ ID NO: 3 - DNA, 19 bp, Synthetic construct
  source 1..19: mol_type = other DNA
atggggaaggtgaaggtcg

SEQ ID NO: 4 - DNA, 22 bp, Synthetic construct
  source 1..22: mol_type = other DNA
ggggtcattgatggcaacaata

SEQ ID NO: 5 - DNA, 24 bp, Synthetic construct
  source 1..24: mol_type = other DNA
acctcattggtgtcactgctactg

SEQ ID NO: 6 - DNA, 21 bp, Synthetic construct
  source 1..21: mol_type = other DNA
tcctcagaatttgggcatcct

(19) United States
(12) Patent Application Publication
(10) Pub. No.: US 2024/0125766 A1
(43) Pub. Date: Apr. 18, 2024

(54) METHOD FOR DETERMINING SUITABILITY OF A SUBJECT TO ANTI TNF ALPHA THERAPY

(71) Applicant: ICHILOV TECH LTD., Tel-Aviv (IL)

(72) Inventors: Ori ELKAYAM, Rishon Lezion (IL); Smadar GERTEL, Herzelia (IL)

(21) Appl. No.: 18/389,949

(22) Filed: Dec. 20, 2023

Related U.S. Application Data
(63) Continuation-in-part of application No. PCT/IL2022/050662, filed on Jun. 20, 2022.
(60) Provisional application No. 63/212,678, filed on Jun. 20, 2021.

(51) Int. Cl. G01N 33/50; C12Q 1/6851; G01N 33/543

(52) U.S. Cl. CPC G01N 33/5008; C12Q 1/6851; G01N 33/54306; G01N 2333/525

(57) ABSTRACT

The present invention is directed to a method for determining the suitability of a subject to TNFα inhibitor therapy. Further provide is a method for treating a subject afflicted with a TNFα related disease.

### REFERENCE TO AN ELECTRONIC SEQUENCE LISTING

The contents of the electronic sequence listing (ICH-P-006-US.xml; size: 6,314 bytes; and date of creation: Nov. 13, 2023) is herein incorporated by reference in its entirety.

### CROSS-REFERENCE TO RELATED APPLICATIONS

This application is a Continuation-in-Part of PCT Patent Application No. PCT/IL2022/050662 having international filing date of Jun. 20, 2022, which claims the benefit of priority of U.S. Provisional Patent Application No. 63/212,678 filed Jun. 20, 2021. The contents of the above applications are all incorporated by reference as if fully set forth herein in their entirety.

### FIELD OF INVENTION

The present invention is in the field of diagnostics, and drug response.

### BACKGROUND

Autoimmune diseases are characterized by systemic inflammation, in which a dysregulated immune system causes damage or dysfunction to target organs. Collectively, autoimmune diseases are estimated to affect over 7% of the general population worldwide. Tumor necrosis factor-alpha (TNFα) is generally considered as the master pro-inflammatory cytokine in autoimmune diseases.

The advent of TNFα inhibitors has dramatically improved the treatment of rheumatoid arthritis (RA), psoriatic arthritis (PsA), ankylosing spondylitis (AS), psoriasis (PsO) and inflammatory bowel disease (IBD). Consequently, TNFa inhibition therapy has become a mainstay treatment for autoimmune diseases in the past two decades. TNFa inhibitors represent the most profitable drug class in history, exceeding 25 billion dollars total per year.

Despite an overall good response to TNFα inhibition therapy, up to 30% of patients are non-responders. Considering the costs and risks involved, biologic agents should be prescribed ideally only to patients in whom therapeutic responses to the drugs are likely to be achieved. Personalized diagnostic tests aim to determine whether an individual patient is likely to benefit from a given therapy. However, currently, TNFα inhibition therapies (e.g., with TNFα inhibitors) are being used in a ‘trial-and-error’ manner, with no prior information or knowledge on the possible response of the patients. Stratifying patients using a diagnostic kit for prediction of response will increase the chance of successful treatment, and will spare the use of ineffective treatments, resulting in an increase in cost-effectiveness which is highly relevant for these expensive drugs. Unfortunately, accurate and clinically validated biomarkers predicting response to TNFα inhibitor are currently lacking.

There are still great needs for in vitro diagnostic kits and methods for accurately determining patient specific response to TNFα inhibition therapy and selecting the optimal therapeutic agent.

### SUMMARY

According to a first aspect, there is provided a method for determining the suitability of a subject to a treatment using a tumor necrosis factor alpha (TNFα ) inhibitor, comprising the steps: (a) providing a sample comprising blood obtained or derived from a subject; (b) contacting the sample with at least one TNFα inhibitor for a period of 72 hours; and (c) determining expression level of interleukin-17 (IL-17) in the sample of step (b), wherein a reduction in the expression level of IL-17 of at least 30% compared to a baseline is indicative of the subject being at least partially responsive for treatment using a TNFα inhibitor, thereby determining the suitability of the subject to a treatment using a TNFα inhibitor.

According to another aspect, there is provided method for treating a subject afflicted with a TNFα related disease, comprising the steps: (a) determining whether an expression level of IL-17 is reduced by at least 55% compared to a baseline, in blood sample obtained or derived from a subject and contacted with at least one TNFα inhibitor for a period of at least 72 hours; and (b) administering to the subject determined as having at least 55% reduction in expression level of IL-17, a therapeutically effective amount of a TNFα inhibitor, thereby treating the subject afflicted with a TNFα related disease.

In some embodiments, a reduction in the expression level of IL-17 of at least 55% at is indicative of the subject being fully responsive for treatment using a TNFα inhibitor.

In some embodiments, the at least one TNFα inhibitor comprises a plurality of types of TNFα inhibitors.

In some embodiments, the blood sample of step (a) is independently contacted with the plurality of types of TNFα inhibitors.

In some embodiments, the method further comprises a step of determining which of the plurality of types of TNFα inhibitors provides the greatest reduction in expression level of IL-17.

In some embodiments, the at least one TNFα inhibitor is an anti-TNFα antibody or a TNFα mimicking receptor.

In some embodiments, the at least one TNFα inhibitor is selected from the group consisting of: Infliximab, Adalimumab, Golimumab, Certolizumab pegol, and Etanercept.

In some embodiments, the subject is afflicted with a TNFα related disease being selected from the group consisting of: psoriatic arthritis, rheumatoid arthritis, ankylosing spondylitis, psoriasis, Crohn's disease, ulcerative colitis, and Uveitis.

In some embodiments, the sample comprises peripheral blood mononuclear cells (PBMCs) derived from the subject.

In some embodiments, the contacting is for a period of at least 72 hours.

In some embodiments, the method further comprises a step preceding step b, comprising extracting RNA from the contacted sample.

In some embodiments, the determining is by quantitative RT-PCR.

In some embodiments, the method further comprises a step preceding step b, comprising extracting proteins from the contacted sample.

In some embodiments, the determining is by an immunoassay.

In some embodiments, the immunoassay is enzyme-linked immunosorbent assay (ELISA).

In some embodiments, the at least one TNFα inhibitor comprises a plurality of types of TNFα inhibitors.

In some embodiments, the blood sample of step (a) is independently contacted with the plurality of types of TNFα inhibitors.

In some embodiments, the method further comprises a step of determining which of the plurality of types of TNFα inhibitors provides the greatest reduction in expression level of IL-17.

In some embodiments, step (b) comprises administering to the subject the TNFα inhibitor determined as providing the greatest reduction in expression level of IL-17.

In some embodiments, the administering comprises intravenously administering or subcutaneously administering.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a non-limiting schematic illustration of assay technique, as disclosed herein. The scheme shows the diagnostic process method for prediction of response to Tumor necrosis factor alpha inhibitors (TNFis).

FIGS. 2A-2C includes vertical bar graphs showing the ability of the herein disclosed assay to determine differential effects of TNFis in an individualized manner. Representative image of assay for all 4 TNFis performed on PBMCs of PsA patients. Each graph shows individual patient results as follows: (2A) A PsA patient that was treated with ETN but could have complete response to all 4 TNFis; (2B) A PsA patient that was a non-responder to ETN and a partial responder to ADA but could have a complete response to IFX and GOL; and (2C) A PsA patient that was non-responder to all TNFis.

FIG. 3 include a graph showing that an analysis of changes in IL-17A expression level in response to TNFis in vitro correlated significantly with the retrospective clinical response. PBMCs derived from PsA n=45 RA n=11 and AS n=7 patients were cultured in vitro with TNFis (ADA, IFX, ETN and GOL) compared with control medium, followed by measurement of the relative expression of IL-17A level. The results were compared with disease outcome in response to each TNFi therapy each patient was treated with. The X axis shows clinical outcome: complete responders (CR), partial responders (PR) and non-responders (NR), and the Y axis shows IL-17A expression level. The p values were calculated by the non-parametric one-way ANOVA Kruskal-Wallis test and Dunn's multiple comparison test.

FIG. 4 includes graphs showing that the relative IL-17A expression in response to each TNFi in the in vitro assay is associated with clinical response to TNFi therapy. The graph shows changes in relative IL-17A expression levels after in-vitro culture with each TNFi corresponding to the agent with which each patient was treated. The results indicate the level of IL-17A expression for each patient, and those values were categorized according to the clinical response: CR, PR and NR.

FIGS. 5A-5C include graphs showing that the accuracy of correlation between assay results and clinical response to ADA, ETN, or both, using the in vitro assay disclosed herein. Statistical model for the evaluation of response to ADA (5A), ETN (5B) or ADA and ETN (5C) using the in-vitro assay.

FIG. 6 includes a graph showing that an analysis of changes in IL-17A expression level in prospective analysis for response to TNFis in vitro correlated significantly with clinical response. PBMCs derived from PsA n=17 and RA n=3 patients were cultured in vitro with TNFis (ADA, IFX, ETN and GOL) compared with control medium, followed by measurement of the relative expression of IL-17A level. The results were compared with prospective disease outcome in response to each TNFi therapy each patient was treated with. The X axis shows clinical outcome: complete responders (CR), partial responders (PR) and non-responders (NR), and the Y axis shows IL-17A expression level. Black dots shows values that fit assay prediction and white dots values that did not fit assay prediction. Thep values were calculated by the non-parametric one-way ANOVA Kruskal-Wallis test and Dunn's multiple comparison test.

FIGS. 7A-7C include graphs showing prospective prediction models for response to ADA, ETN or both using the in vitro assay. Statistical model for the evaluation of prospective response to ADA (7A), ETN (7B), or ADA and ETN (7C) using the in-vitro assay disclosed herein.

FIG. 8 includes a vertical bar graph showing two different time points to assess the in vitro ETN effect on IL-17A expression level in PBMCs derived from a TNFi-responder PsA patient, after 24 hr and after 72 hr.

FIGS. 9A-9B include graphs showing that ADA, IXE and TCZ differentially modulate IL-17A mRNA expression in the in vitro assay in PBMCs derived from PsA and RA patients. PBMCs were co-cultured in the presence of ADA, IXE, TCZ or medium alone as a control. After 72 h of incubation, RNA was extracted and real-time PCR for quantification of IL-17A was performed. (9A) PBMCs derived from PsA patients (n=70). (9B) PBMCs derived from RA patients (n=27).

FIG. 10 includes a graph showing that retrospective and prospective validation of the diagnostic assay results correlated with clinical response. Assay results for IL-17A expression in PsA n=68, RA n=16 and AS n=9 patients before TNFi therapy. The X axis shows clinical outcome: complete responders (CR), partial responders (PR) and non-responders (NR), and the Y axis shows IL-17A expression level. The p values were calculated by the non-parametric one-way ANOVA Kruskal-Wallis test and Dunn's multiple comparison test.

FIGS. 11A-11B include vertical bar graph showing comparison of IL-17A at mRNA and protein levels in response to TNFi in the bioassay. IL-17A mRNA level were measured by qRT-PCR (11A); and IL-17A protein levels were measured by enzyme-linked immunosorbent assay (ELISA) (11B).

### DETAILED DESCRIPTION

According to another aspect, there is provided method for treating a subject afflicted with a TNFα related disease, comprising the steps: (a) determining whether an expression level of IL-17 is reduced compared to a baseline, in blood sample obtained or derived from a subject and contacted with at least one TNFα inhibitor for a period of at least 72 hours; and (b) administering to the subject determined as having a reduction in expression level of IL-17, a therapeutically effective amount of a TNFα inhibitor, thereby treating the subject afflicted with a TNFα related disease.

According to some embodiments, there is provided a method for determining the suitability of a subject to a treatment using a tumor necrosis factor alpha (TNFα) inhibitor, comprising the steps: (a) providing a sample obtained or derived from a subject; (b) contacting the sample with at least one TNFα inhibitor for a period of at least 48 hours; and (c) determining expression level of interleukin-17 (IL-17) in the sample of step (b).

In some embodiments, the sample is contacted with at least one TNFα inhibitor for a period of at least 36 hours, at least 40 hours, at least 48 hours, at least 52 hours, at least 60 hours, at least 66 hours, at least 72 hours, at least 78 hours, at least 82 hours, or any value and range therebetween. Each possibility represents a separate embodiment of the invention. In some embodiments, the sample is contacted with at least one TNFα inhibitor for a period of 36-80 hours, 40-72 hours, 48-66 hours, 52-96 hours, 60-84 hours, 66-78 hours, or 64-78 hours. Each possibility represents a separate embodiment of the invention.

In some embodiments, the method comprises contacting a sample with at least one TNFα inhibitor for a period of at least 36 hours, at least 40 hours, at least 48 hours, at least 52 hours, at least 60 hours, at least 66 hours, at least 72 hours, at least 78 hours, at least 82 hours, or any value and range therebetween. Each possibility represents a separate embodiment of the invention. In some embodiments, the method comprises contacting a sample with at least one TNFα inhibitor for a period of 36-80 hours, 40-72 hours, 48-66 hours, 52-96 hours, 60-84 hours, 66-78 hours, or 64-78 hours. Each possibility represents a separate embodiment of the invention.

In some embodiments, a reduction in the expression level of IL-17 of at least 5%, at least 10%, at least 15%, at least 20%, at least 30%, at least 40%, at least 45%, or at least 50% compared to a baseline, or any value and range therebetween, is indicative of the subject being at least partially responsive to a treatment using a TNFα inhibitor. Each possibility represents a separate embodiment of the invention.

In some embodiments, a reduction in the expression level of IL-17 of at least 51%, at least 55%, at least 60%, at least 70%, at least 80%, at least 90%, at least 95%, at least 99%, or 100% compared to a baseline, or any value and range therebetween, is indicative of the subject being responsive or fully responsive to a treatment using a TNFα inhibitor. Each possibility represents a separate embodiment of the invention.

In some embodiments, a reduction in the expression level of IL-17 of at most 1%, 2% at most, 5% at most, 7% at most, 8% at most, 9% at most, 10% at most, or 15% at most, compared to a baseline, or any value and range therebetween, is indicative of the subject being non-responsive to a treatment using a TNFα inhibitor. Each possibility represents a separate embodiment of the invention.

In some embodiments, no reduction or increase in the expression level of IL-17 compared to a baseline is indicative of the subject being non-responsive to a treatment using a TNFα inhibitor.

As used herein, the terms “Interleukin-17” or “IL-17” refer to a pro-inflammatory cytokine, known to be produced by T helper cells.

In some embodiments, IL-17 is selected from: IL-17A, IL-17B, IL-17C, IL-17D, IL-17E, IL-17F, any combination thereof, or any functional analog thereto having at least 15%, 20%, 40%, 50%, 60%, 70%, 80%, 90%, 95%, 97%, or 99% sequence identity or homology thereto. In some embodiments, IL-17 is or comprises IL-17A.

In some embodiments, a functional analog encompasses any peptide or protein characterized by having an expression level being modifiable essentially similar to IL-17, in the context of an anti TNFα agent or a TNFi, according to the method disclosed herein.

In some embodiments, essentially similar comprises 1% different at most, 2% different at most, 3% different at most, 5% different at most, 7% different at most, 10% different at most, 15% different at most, 20% different at most, 25% different at most, 35% different at most, or any value and range therebetween. Each possibility represents a separate embodiment of the invention.

As used herein, the term “baseline” encompasses a control sample. In some embodiments, a control comprises uncontacted or untreated blood. In some embodiments, a control comprises uncontacted or untreated PBMC. In some embodiments, a control comprises a sample derived or obtained from a healthy subject. In some embodiments, a control comprises uncontacted or untreated blood, PBMC, or both, derived or obtained from a healthy subject.

In some embodiments, a sample is a biological sample. In some embodiments, a sample is a biological sample obtained or derived from a subject. In some embodiments, a sample comprises a blood sample obtained or derived from a subject. In some embodiments, a sample comprises peripheral blood mononuclear cells (PBMC) obtained or derived from a subject. In some embodiments, a blood sample comprises PBMC obtained or derived form a subject.

Methods for isolating or obtaining a sample from a subject would be apparent to a skilled physician. Non-limiting example for obtaining PBMC is exemplified herein.

According to some embodiments, there is provided a method for treating a subject afflicted with a TNFα related disease, comprising the steps: (a) determining whether an expression level of IL-17 is reduced by at least 51%, at least 55%, at least 60%, at least 70%, at least 80%, at least 90%, at least 95%, at least 99%, or 100% compared to a baseline, or any value and range therebetween, in a sample obtained or derived from a subject and contacted with at least one TNFα inhibitor for a period of at least 36 hours, at least 40 hours, at least 48 hours, at least 52 hours, at least 60 hours, at least 66 hours, at least 72 hours, at least 78 hours, at least 82 hours, or any value and range therebetween; and (b) administering to the subject determined as having at least 51%, at least 55%, at least 60%, at least 70%, at least 80%, at least 90%, at least 95%, at least 99%, or 100% compared to a baseline, or any value and range therebetween, reduction in expression level of IL-17, a therapeutically effective amount of a TNFα inhibitor. Each possibility represents a separate embodiment of the invention.

In some embodiments, at least one TNFα inhibitor comprises a plurality of types of TNFα inhibitors. In some embodiments, at least one TNFα inhibitor comprises a plurality of types of different TNFα inhibitors.

As used herein, the term “plurality” comprises any integer equal to or greater than 2. In some embodiments, a plurality of types of TNFα inhibitors comprises 2-3, 2-4, 2-5, 3-4, 3-5, or 4-5 types of TNFα inhibitors.

In some embodiments, a TNFα inhibitor is an anti-TNFα antibody or a TNFα mimicking receptor.

In some embodiments, a TNFα inhibitor is selected from: Infliximab, Adalimumab, Golimumab, Certolizumab pegol, Etanercept, or any combination thereof.

In some embodiments, the plurality of types of TNFα inhibitors comprises or is selected from: Infliximab, Adalimumab, Golimumab, Certolizumab pegol, Etanercept, or any combination thereof.

As used herein, the term “TNF a inhibitor” encompasses any compound known to one of ordinary skill in the art to be characterized by an inhibitory effect over TNFα, and signalling thereof. In some embodiments, the TNFα inhibitor comprises enantiomers, analogs, derivatives, bio similarities, of any TNFα inhibitor, which are common and would be apparent to one of ordinary skill in the art.

In some embodiments, the TNFα comprises an antagonist of a TNFα receptor. In some embodiments, TNFα comprises a steroid, a small molecule, or any combination thereof.

In some embodiments, the sample of step (a), according to the herein disclosed method, is independently contacted with a plurality of types of TNFα inhibitors, as described herein.

In some embodiments, the method further comprises a step of determining which of the plurality of types of TNFα inhibitors provides the greatest reduction in expression level of IL-17. In some embodiments, the method further comprises independently comparing the expression level of IL-17 contacted with any one of the plurality of types of TNFα inhibitors.

In some embodiments, the method comprises administering to the subject the TNFα inhibitor of the plurality of types of TNFα inhibitors which was determined as providing the greatest reduction in expression level of IL-17.

In some embodiments, administering comprises intravenously administering or subcutaneously administering.

In some embodiments, the subject is afflicted with a TNFα related disease.

As used herein, the term “TNFα related disease” refers to any disease, condition, disorder, pathology, or any combination thereof, wherein TNFα is involved, induces, initiates, propagates, determines, or any combination or equivalent thereof, in the pathogenesis, pathophysiology, or both.

In some embodiments, a TNFα related disease is selected from: psoriatic arthritis, rheumatoid arthritis, ankylosing spondylitis, psoriasis, Crohn's disease, and ulcerative colitis.

In some embodiments, expression comprises gene expression levels, such as, mRNA transcription levels or an equivalent thereof. In some embodiments, expression comprises protein expression levels. In some embodiments, expression comprises gene and protein expression levels.

In some embodiments, the method further comprises a step comprising extracting RNA from the contacted sample. In some embodiments, the method further comprises a step comprising extracting at least one protein from the contacted sample.

In some embodiments, extracting comprises extracting RNA from blood, PBMCs, or the like as disclosed herein.

In some embodiments, extracting comprises extracting at least one protein from a medium or a suspension comprising blood, PBMCs, or the like as disclosed herein. In some embodiments, extracting at least one protein does not involve or include homogenizing and/or lysing the blood, PBMCs, or the like as disclosed herein.

Methods for RNA extraction are common and would be apparent to one of ordinary skill in the art of molecular biology, such as exemplified herein.

Methods for extracting protein(s) are common and would be apparent to one of ordinary skill in the art of biochemistry, such as exemplified herein.

In some embodiments, the extracting is performed after the sample being contacted with at least one TNFα inhibitor. In some embodiments, the extraction step precedes the determining step of the herein disclosed method. In some embodiments, the extraction step precedes the step (b) of the herein disclosed method.

In some embodiments, determining is by quantitative RT-PCR (qRT-PCR). In some embodiments, determining is by next generation sequencing. In some embodiments, qRT-PCR comprises relative qRT-PCR or absolute qRT-PCR.

As used herein, the term “immunoassay” encompasses any bioanalytical method which provides quantitation of a molecule of interest, e.g., an analyte, that depends on or utilizes recognition of the analyte/molecule of interest/antigen by an antibody.

In some embodiments, determining is by an immunoassay. In some embodiments, an immunoassay comprises any antibody-based assay used for protein detection, quantification, or both. In some embodiments, determining comprises an enzyme-linked immunosorbent assay (ELISA). In some embodiments, an immunoassay comprises: ELISA, immunoblot, dot blot, western blot, or the like.

In some embodiments, the determining comprises in vitro determining. In some embodiments, in vitro refers to any condition outside the body of a living organism. In some embodiments, in vitro is in a plate, a tube, or any equivalent thereof, suitable for determining gene expression, according to the herein disclosed method.

In some embodiments, the subject is a mammal. In some embodiments, the subject is a primate. In some embodiments, the subject is a human subject.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

### General

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells - A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Peripheral Blood Mononuclear Cell (PBMC) Purification and Cell Culture

Samples of peripheral venous blood from patients were collected in EDTA pre-coated tubes. PBMC were isolated from EDTA-blood using Lymphoprep™ (Axis-Shield, Oslo, Norway). Cells were cultured at a density of 2×10⁶ cells/ml in a 48-well plate in RPMI 1640 medium containing 10% fetal calf serum supplemented with penicillin (100 U/mL), streptomycin (100 μg/mL), 2 mmol/L L-glutamine, and 50 μM β-mercaptoethanol.

Cells were incubated for 72 h at 37° C. with TNFα blocker (adalimumab, ADA at a concentration of 10 μg/ml, these doses reflect the biologic agents' concentrations in human serum). Sample without a TNFα inhibitor (medium alone) is used as a control.

RNA extraction

At the end of the incubation period cells were collected and lysed with lysis buffer from the total RNA extraction kit (High Pure RNA Isolation Kit product no. 11828665001). Samples were either frozen at −20° C. or the RNA extraction process was completed according to the manufacturer's instructions. As a final step, the RNA is eluted in DEPC water (a total volume of 37 μl RNA was extracted).

Finally, RNA concentration was determined using the NanoDrop spectrophotometer ND-8000 (Thermo Scientific, Braunschweig, Germany). The extracted total RNA was reverse transcribed to cDNA immediately, or stored at −80° C.

### cDNA synthesis

Three hundred (300) ng of total RNA for each sample was transcribed into cDNA using the High-Capacity cDNA Reverse Transcription Kit (Life Technologies, Carlsbad, CA), with random hexamers, according to the manufacturer's protocol.

### Oligonucleotide Primers

Primers for amplification of interleukin 17 (IL-17) and the housekeeping gene glyceraldehyde 3-phosphate dehydrogenase (GAPDH), were designed based on the sequences deposited in GenBank. Primers length was restricted to 18-24 bp and amplification product size was from 100-300 bp. The primers used are provided in Table 1.

TABLE 1
Primers

| | Accession | | |
|---|---|---|---|
| | no. of | Sequence | Position |
| | gene | (SEQ ID | on |
| Primer name | target | NO:) | mRNA |
| Human_IL-17A_F | NM_002190 | TGTCACT | 83-102 |
| | | GCTACTG | |
| | | CTGCTG | |
| | | (1) | |
| Human_IL-17A_R | | GTGAGGT | 274-255 |
| | | GGATCGG | |
| | | TTGTAG | |
| | | (2) | |
| Human_IL-17A_D_F | | ACCTCAT | |
| | | TGGTGTC | 73-96 |
| | | ACTGCTA | |
| | | CTG | |
| | | (5) | |
| Human_IL-17A_D_R | | TCCTCAG | 170-150 |
| | | AATTTGG | |
| | | GCATCCT | |
| | | (6) | |
| Human_GAPDH_F_A | NM_002046 | ATGGGGA | 77-95 |
| | | AGGTGAA | |
| | | GGTCG | |
| | | (3) | |
| Human_GAPDH_R_A | | GGGGTCA | |
| | | TTGATGG | 184-163 |
| | | CAACAAT | |
| | | A | |
| | | (4) | |

### RT-qPCR

Quantitative PCR was performed on the StepOnePlus Real-Time PCR System (Applied Biosystems). Reactions were performed in 96-well plates; each well contained 10 μl of solution. For each reaction 5 ng cDNA per well that was added to 2×Fast SYBR® Green dye mix (Applied Biosystems), 7 pmol of each primer and DEPC nuclease free water. The thermal cycling program of the qPCR was one cycle at 95° C. for 10 min, followed by 50 cycles of 95° C. for 15 sec and 60° C. for 1 min. A dissociation step was also included to confirm the specificity of amplification. Three technical replicates of each biological sample were run, and negative template control was also included in the qRT-PCR reaction.

### Data Analysis Using the 2^{-ΔΔ} Ct Method

Analysis of relative gene expression was carried out with the comparative threshold cyclers (Ct) method also referred to as the 2^{-ΔΔ} method. In brief, relative quantification analysis determines the levels of expression of a gene of interest (GOI) and expresses it relative to the levels of an internal control or Reference Gene (RefG). The following shorthand is used: ΔΔCt=ΔCt (Calibrator)−ΔCt (Sample); ΔCt (Calibrator)=Ct (GOI, Calibrator)−Ct (RefG, Calibrator); and ΔCt (Sample)=Ct (GOI, Sample)−Ct (RefG, Sample). The calibrator is a control sample without addition of a TNFα inhibitor. Reference genes are genes that are not affected by the treatment in any way and are constant under the tested conditions, such as the housekeeping gene GAPDH.

### Assay Performance and Results Analysis

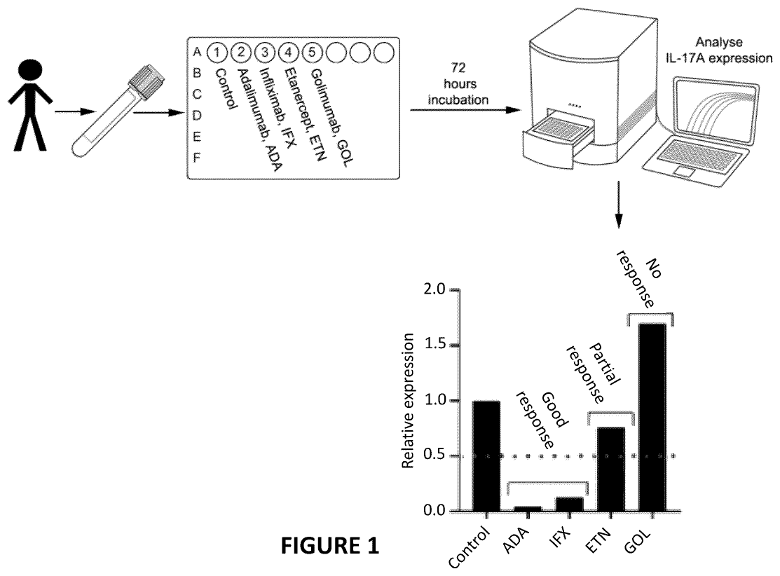

The herein disclosed assay is based on a single blood sample derived from patients that are candidates to initiate/change TNFi therapy. PBMCs are extracted and co-cultured in vitro with a panel of TNFis, after which qPCR is performed to assess the changes in IL-17A gene expression. Of note, all immune cell populations involved in TNFi signaling that eventually lead to response or non-response are present in the culture. TNFis are supplemented to the in vitro culture at physiological concentrations (reflecting doses of TNFis in a patient's circulation). A sample with medium alone (without TNFis) is used as a reference control. PBMCs are co-cultured for 72 h, after which RNA is extracted and real-time PCR is performed on cDNA to analyze IL-17A expression levels. The expression level in response to each TNFi is then compared to the expression in the control (without TNFi). For each patient relative expression in the reference control receives value of≈1. Down-regulation (value below 0.5) in the relative IL-17A expression following co-culture with TNFi is considered as good-response. Up-regulation (above a value of 0.5 or an approximate value around 0.5) in the relative IL-17A expression following co-culture with TNFi is considered as a non-response. The innovative method overview is shown in FIG. 1.

### ELISA for IL-17A

An enzyme-linked immunosorbent assay (ELISA) was used to measure the concentration of IL-17A in culture supernatants. IL-17A in the supernatants was measured with an Duoset ELISA for Human IL-17A (R&D systems).

### EXAMPLE 1

### Personalized Detection of Response to TNFis and Association with Clinical Response

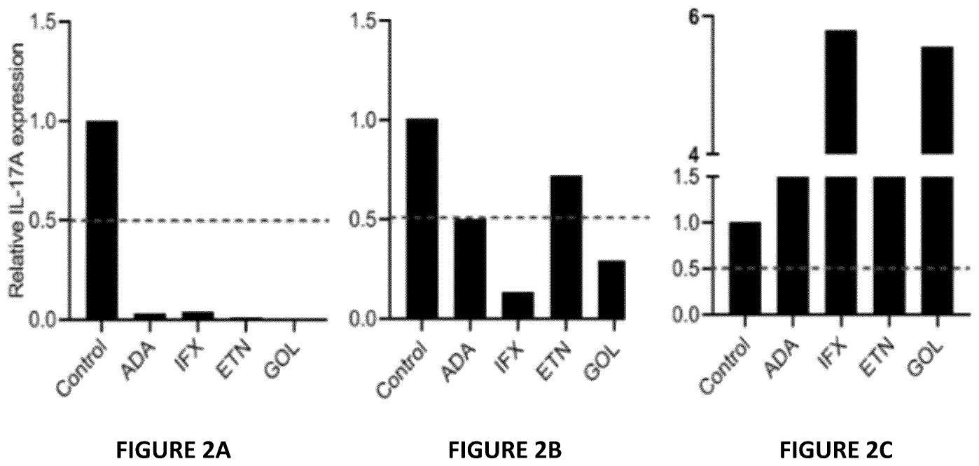

The in vitro assay was performed on PBMCs derived from patients treated with one or more TNFis and their outcome clinical response was recorded. Since for each patient there is unique response to the different TNFis, the inventors aimed at analyzing the patient's cells response to the various TNFis. The assay analyzed the differential impacts of the 4 available TNFis [adalimumab (ADA), infliximab (IFX), etanercept (ETN) and golimumab (GOL] and the in-vitro results for the ability of each TNFi to modulate the IL-17A expression level were correlated to the recorded therapeutic response to specific TNFis with which the patients were treated. A representative image of the results is shown in FIG. 2. The graph in FIG. 2A represents the results for an ETN-treated patient, and the IL-17A level can be seen to have been markedly reduced in that ETN sample. Moreover, the results indicate that this patient could have good response to the other available TNFis (i.e., ADA, IFX and GOL). FIG. 2B displays representative results for a patient that was treated with two different TNFis, ETN with no response and ADA with partial response. The figure shows that the effect of ETN and ADA could be reflected by the clinical response in the form of a high IL-17A expression level for ETN and a median IL-17A expression level for ADA. In addition, IFX and GOL can be seen to reduce the IL-17A expression level to a greater extent in this patient. FIG. 2C provides representative results for a patient that was treated with the four TNFis and found to be non-responder to all of them. The results clearly demonstrate that incubation with all TNFis resulted in elevated IL-17A expression levels that indicate non-response to each TNFis.

The preliminary data indicate that our in vitro diagnostic assay could be used as a sensitive tool to analyze the differential effect of TNFis on IL-17A expression, and that it could be used to predict response to TNFis in an individualized manner.

EXAMPLE 2

### Correlations between changes in IL-17A expression levels in response to TNFis and clinical outcomes

In a retrospective analysis the inventors initially assessed the clinical utility of the in vitro assay for its ability to predict the clinical response to ADA therapy (n=31) and then elaborated the study for patients that were treated with all the 4 TNFis (ADA, IFX, ETN and GOL) (n=27). The results of the IL-17A expression level following in vitro incubation with all 4 TNFis were correlated with the patient's response to each TNFis which each patient was treated with. The analysis was performed on PBMCs derived from 44 patients with PsA, 9 patients with RA and 5 patients with AS whose clinical data and response to TNFis therapy are shown in Table 2.

TABLE 2
Characteristics of patients with PsA, RA and AS in the retrospective study.

| | | PsA (n = 44) | RA (n = 9) | | AS (n = 5) | |
|---|---|---|---|---|---|---|
| Age, (years) | | 52.4 ± 2.0 | 67.2 ± 3.2 | | 41.0 ± 1.6 | |
| Gender | | 21/23 | 8/1 | | 2/3 | |
| (Female/male) | | | | | | |
| Current | TNFi | Adalimumab, | TNFi | Adalimumab, | TNFi | Adalimumab, |
| biologic | | n = 8 | | n = 2 | | n = 2 |
| therapy | | Etanercept, | | Infliximab, | | Etanercept, |
| | | n = 7 | | n = 3 | | n = 1 |
| | | Infliximab, | | | | Infliximab, |
| | | n = 4 | | | | n = 1 |
| | | Golimumab, | | | | Golimumab, |
| | | n = 3 | | | | n = 1 |
| | | Certolizumab | | | | |
| | | pegol, n = 1 | | | | |
| | IL-17Ai | Secukinumab, | | | | |
| | | n = 13 | | | | |
| | | Ixekizumab, | | | | |
| | | n = 4 | | | | |
| | IL-23i | Guselkumab, | | | | |
| | | n = 1 | | | | |
| | JAK | Tofacitinib. | JAK | Tofacitinib. | | |
| | inhibitor | n = 1 | inhibitor | n = 1 | | |
| | PDE4 | Apremilast. | IL-6R | Tocilizumab, | | |
| | inhibitor | n = 1 | inhibitor | n = 2 | | |
| | Untreated | n = 1 | CD20 | Rituximab, | | |
| | | | inhibitor | n = 1 | | |

| Response to | Complete responders (CR) 26/58 (45%) |
|---|---|
| TNFis | Partial responders (PR) 12/58 (21%) |
| | Non-responders (NR) 20/58 (34%) |

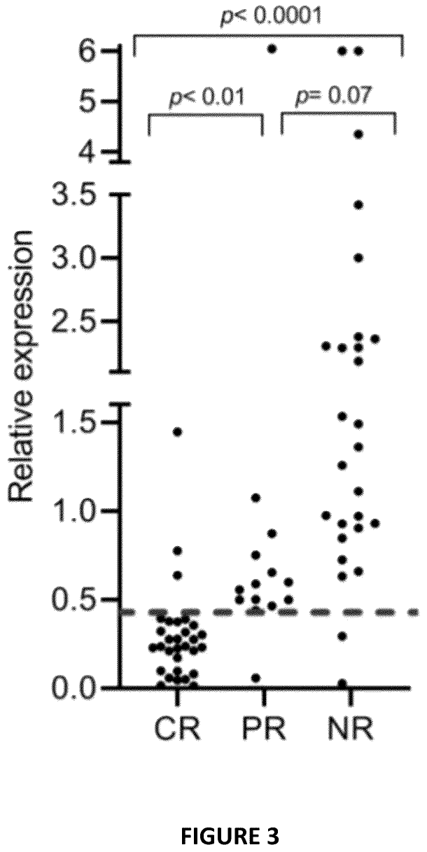

The assay was performed on PBMCs derived from patients with clinical details as described in Table 1. PBMCs were incubated with medium (reference control) or with each one of the 4 TNFis, followed by determination of the IL-17A mRNA expression level. The clinical response of patients to TNFis therapy was categorized as complete response (CR), partial response (PR) and no response (NR). Among the 58 patients, five patients were treated with more than one TNFis (did not respond to one TNFi and then switched to another) their results for response for each TNFi were correlated with the in vitro results (total number 66 results). IL-17A mRNA was differentially expressed, and its expression level correlated with response to TNFis therapy each patient was treated with, as shown in FIG. 3.

The mean IL-17A mRNA expression level was significantly low in CR (0.29±0.05) as compared to PR (1.01±0.42) and NR (2.0±0.45). The difference between the IL-17A expression level in response to each TNFi correlated significantly with clinical response between CR and NR (p<0.0001) and between CR and PR (p=0.02), however, the difference between PR and NR was not significant. The retrospective accuracy of the assay was 92.4% (61 correct results out of 66) (FIG. 3).

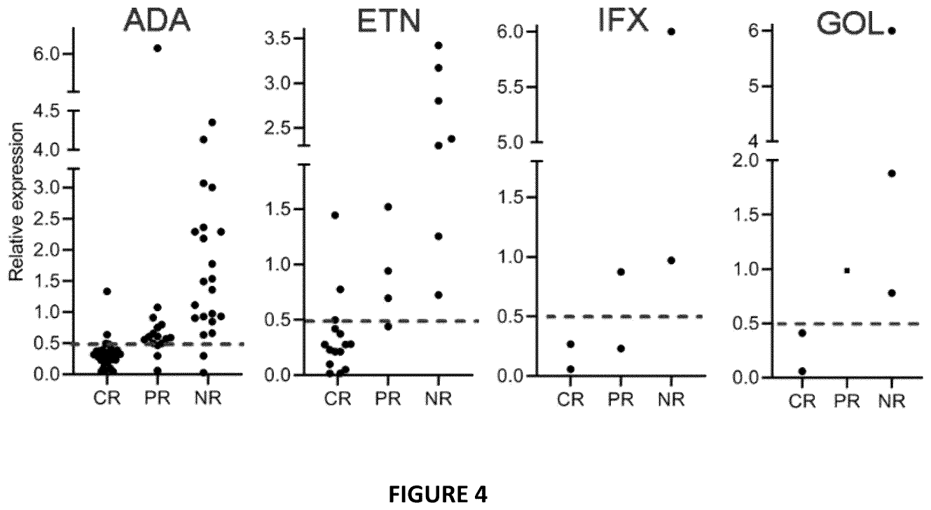

Furthermore, the expression of the IL-17A level in the assay was analyzed separately for each TNFi for the 3 clinical response groups (FIG. 4).

### EXAMPLE 3

### Statistical model for calculation of retrospective accuracy for determination of IL-17A expression levels with clinical response to the specific TNFi

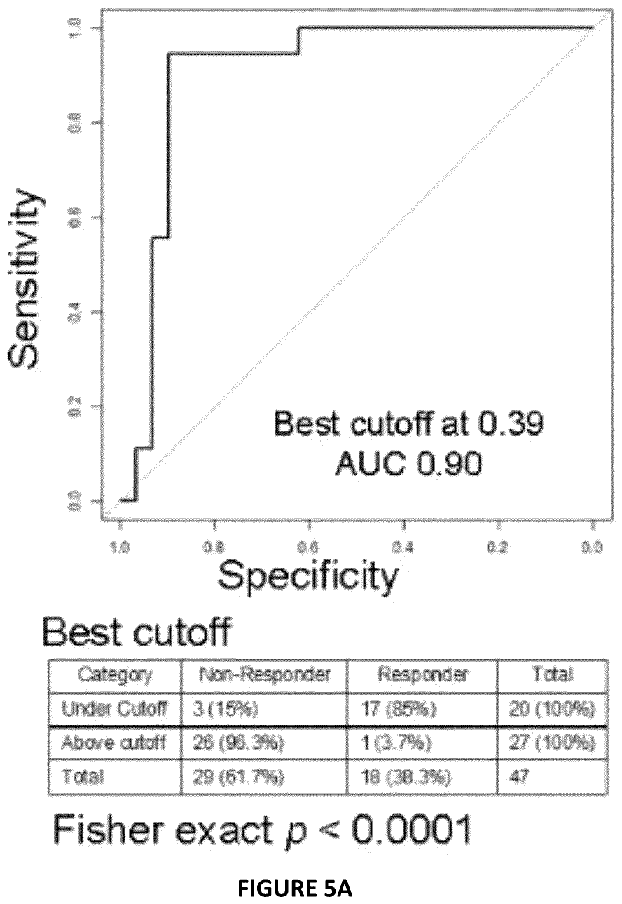

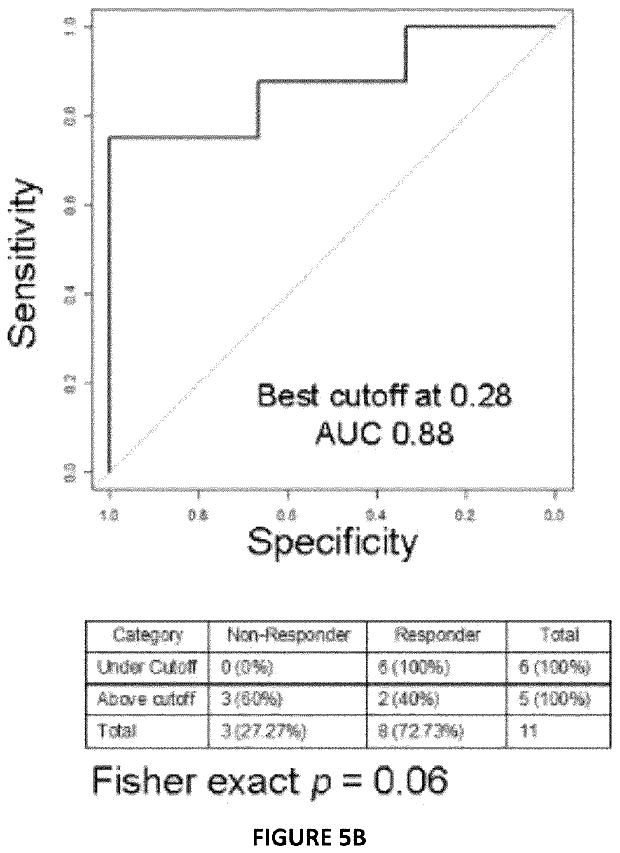

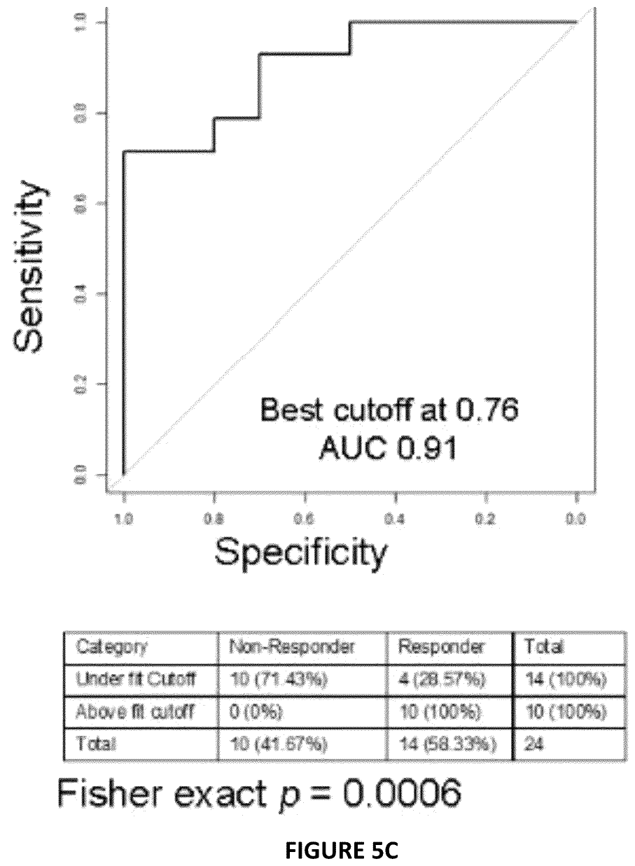

Patients participating in this cohort were either treated with ADA and ETN therapy and the accuracy power of analyzing changes in IL-17A expression levels for correlation of response to ADA or ETN or both using the assay was performed. The following comparisons were performed in order to analyze the ability of this assay to predict response to ADA (FIG. 5A), ETN (FIG. 5B) or either TNFis (ADA or ETN) (FIG. 5C). The response to ADA showed a positive prediction value of 85% (under the cut-off) and a negative prediction value of 96.3% (above the cut-off). This means that if a subject reaches an IL-17A expression value below 0.394, the model predicts 85% success and above 0.394 96% failure for ADA treatment (FIG. 5A). Predicting the response to ETN showed a positive prediction value of 100% (under the cut-off) and a negative prediction value of 60% (above the cut-off). Thus, if a subject reaches an ETN expression value below 0.276, the model predicts success for ETN treatment of 100% (FIG. 5B). A model that used both ADA and ETN for the accuracy of any response showed a positive prediction value of 71.4% (under cut-off, meaning response) and a negative prediction value of 100% (above cut-off meaning non-response) (FIG. 5C). The cutoff in FIG. 5C is in fit values (arbitrary units) of the model and is not comparable to the gene expression of FIGS. 5A-5B.

### EXAMPLE 4

### Prospective analysis of the assay for determination of assay accuracy for personalized selection of TNFis

The inventors then performed a prospective assessment in order to determine the ability of the assay to predict the response to 4 available TNFis. The cohort included 20 patients (PsA n=17 and RA n=3) treated with at least 1 of the 4 TNFis (one patient was treated with one and then switch to another). Their clinical data and responses to TNFis are shown in Table 3.

TABLE 3
Characteristics of patients with PsA and RA in the study cohort.

| | PsA (n = 17) | | RA (n = 3) | |
|---|---|---|---|---|
| Mean age (years) | 55.9 ± 2.5 | | 52.3 ± 4.1 | |
| Gender (Female/male) | 8/9 | | 3/0 | |
| Current biologic | TNFi | Adalimumab, n = 7 | TNFi | Adalimumab, |
| therapy | | | | n = 1 |
| | | | | Etanercept, |
| | | | | n = 1 |
| | | Etanercept, n = 3 | | Infliximab, |
| | | Golimumab, n = 3 | | n = 1 |
| | | Infliximab, n = 1 | | |
| | IL-17Ai | Secukinumab, n = 1 | | |
| | | (due to no- | | |
| | | response to TNFi | | |
| | | therapy) | | |
| | | Ixekizumab, n = 2 | | |

| | |
|---|---|
| Response to TNFis | Complete responders (CR) 10/20 (50%) |
| | Partial responders (PR) 4/20 (20%) |
| | Non-responders (NR) 6/20 (30%) |

### EXAMPLE 5

### Schematic Illustration of Assay Potential to Predicts Future Clinical Response to TNFis in Prospective Analysis

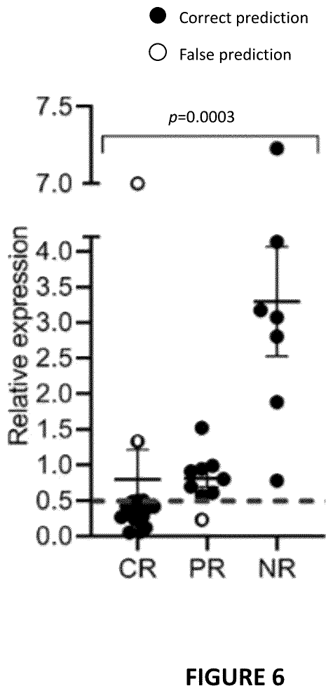

Prior to treatment, the inventors analyzed using the assay response to TNFis and correlated the results against prospective clinical response to TNFis in 20 patients (PsA n=17 and RA n=3 patients, one patient was treated prospectively with 2 TNFis, initially with ETN and after partial response switch to ADA). FIG. 6 shows the values of IL-17A expression that represent as dots of their assay results to the specific TNFi which they were treated with and categorized according to the clinical response: CR, PR and NR. Black dots show values that fit assay prediction and white dots values that did not fit assay prediction.

The mean IL-17A mRNA expression level was significantly low in CR (0.40±0.35) as compared to PR (0.72±0.62) and NR (2.44±0.81). The difference between the IL-17A expression level in response to each TNFi correlated significantly with clinical response between CR and NR (p<0.03). The prospective prediction of the assay was 90.5% (19 correct results out of 21, one patient among the 20 was treated with 2 TNFis) (FIG. 6). The prospective analysis indicates on the ability to predict both effective and ineffective TNFis treatment and provides preliminary clinical demonstration of the validated assay as a powerful tool for personalized prediction of response to TNFis.

### EXAMPLE 6

Statistical model for prediction of prospective response to the specific TNFi by means of the in vitro assay

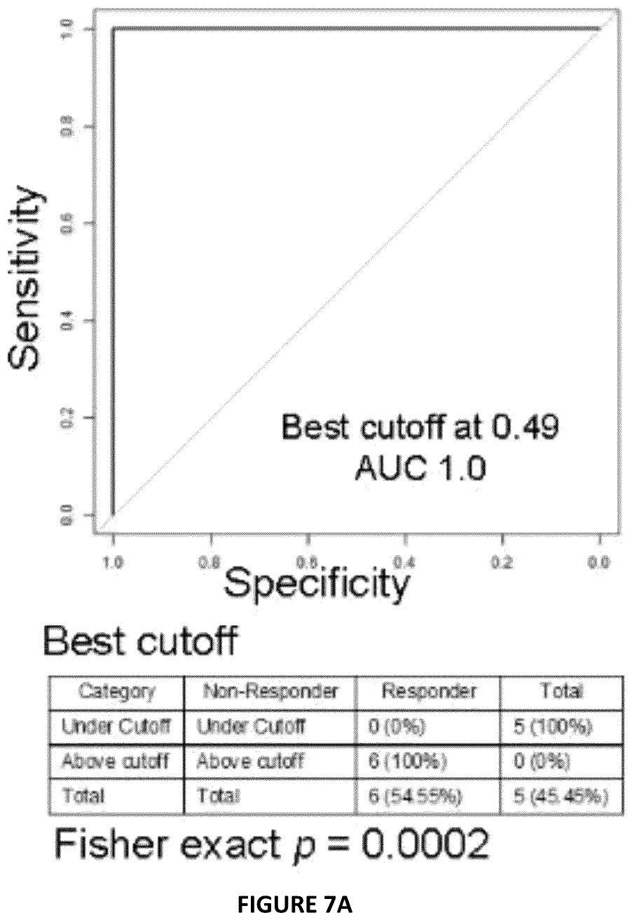

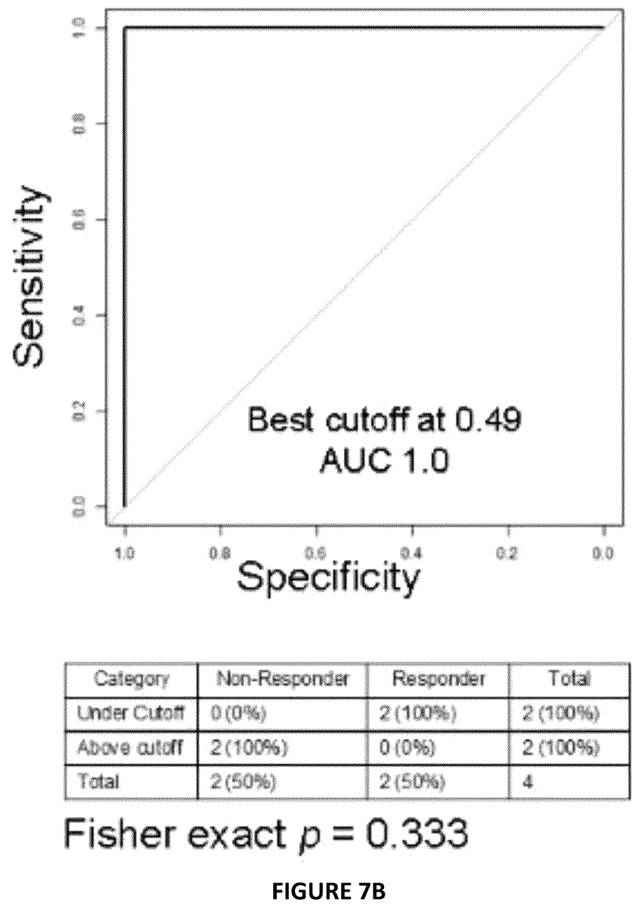

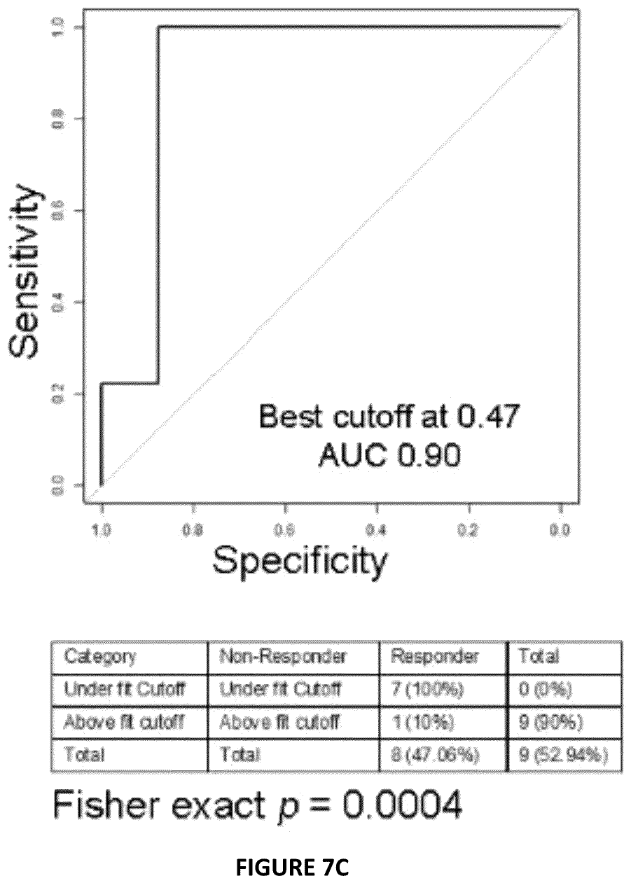

Patients participating in this cohort were either treated with ADA and ETN therapy and the predictive power of analyzing changes in IL-17A expression levels for predicting the response to ADA or ETN or both using the assay was performed. The following comparisons were performed in order to analyze the ability of this assay to predict response to ADA (FIG. 7A), ETN (FIG. 7B) or either TNFis (ADA or ETN) (FIG. 7C). The response to ADA showed a positive prediction value of 100% (under the cut-off) and a negative prediction value of 100% (above the cut-off). This means that if a subject reaches an ADA expression value below 0.49 the model predict a 100% success and above 0.49 a 100% failure for ADA treatment (FIG. 7A). Predicting the response to ETN showed a positive prediction value of 100% (under the cut-off) and a negative prediction value of 100% (above the cut-off). Thus, if a subject reaches an ETN expression value below 0.497, the model predicts success for ETN treatment of 100% (FIG. 7B). A model that used both ADA and ETN for the prediction of any response showed significant divergence, with a positive prediction value of 90% (under cut-off, meaning response) and a negative prediction value of 100% (above cut-off meaning non-response) (FIG. 7C). The cutoff in FIG. 7C is in fit values of the model and is not comparable to the gene expression of FIGS. 7A-7B.

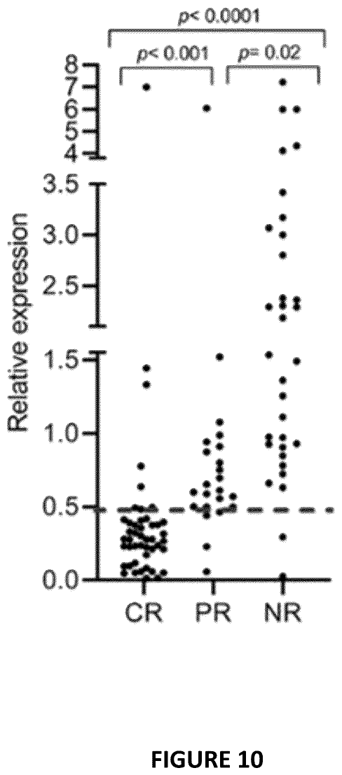

Further, the expression of the IL-17A level in the assay was analyzed for the entire study in CR, PR and NR (FIG. 10).

The mean IL-17A mRNA expression level was significantly low in CR (0.46±0.15) as compared to PR (0.91±0.25) and NR (2.2±0.29). The difference between the IL-17A expression level in response to each TNFi correlated significantly with clinical response between CR and NR (p<0.0001) and between CR and PR (p<0.001), difference between PR and NR (p=0.02). The all-study accuracy of the assay was 90% (94 correct results out of 104).

EXAMPLE 7

### Determination of an Incubation Timeframe to Measure Optimal Effects of TNFis on Cellular Response in the In Vitro Assay

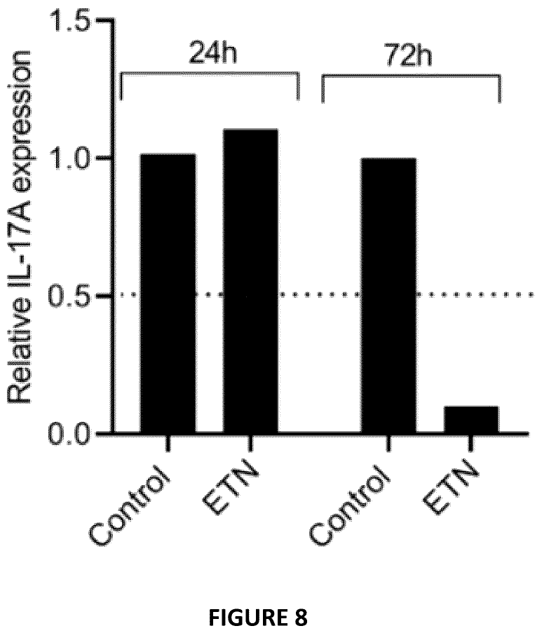

The optimal incubation period to determine the effect of TNFi on cellular response and modulation of IL-17A expression in the vitro assay was assessed at two time points (24 h and 72 h). PBMCs were extracted from a TNFi (ETN)-responder PsA patient, the assay was terminated at each time point, and the effect of the TNFi on IL-17A expression was determined. IL-17A mRNA expression at each time point was determined for controls vs a given TNFi (Etanercept, ETN) (FIG. 8). After 24 h of incubation, ETN did not reduce IL-17A expression compared to the control, whereas IL-17A expression was down-regulated as expected after 72 h.

### EXAMPLE 8

### Specificity of the In Vitro Assay for Prediction of Response to TNFis

The inventors examined whether the in vitro assay for prediction of response to TNFis could be utilized for additional biologic agents with different mechanisms of action. TNFis are effective drugs for the treatment of PsA and RA. IL-17 inhibitors bring a clear benefit in the reduction of disease symptoms of PsA and AS, but they are not recommended to treat RA. On the other hand, while interleukin-6 receptor (IL-6R) blockers have emerged as effective drugs for RA, their effect in PsA has been disappointing. The inventors tested whether modulation of IL-17A mRNA expression is different following incubation with TNF as well as with IL-17A and IL-6R inhibitors using the in vitro assay in PBMCs derived from PsA and RA patients. Cells were incubated for 72 hours with TNFi (adalimumab, ADA), IL-17Ai (Ixekizumab, IXE), and IL-6Ri (tocilizumab, TCZ) or medium alone as control. RNA was extracted at the end of incubation, and determination of IL-17A mRNA expression was performed.

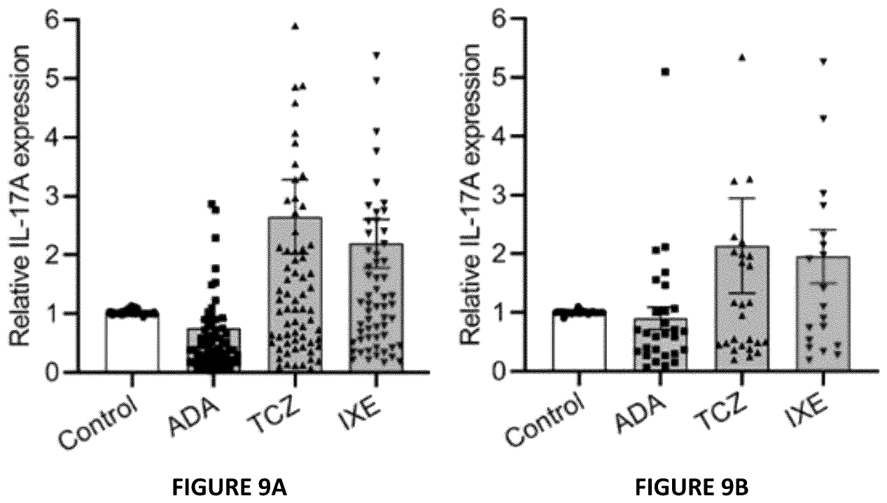

ADA reduced the average IL-17A expression as expected in 60-70% responders among the PsA and RA patients (FIG. 9). However, IL-17A and IL-6R inhibitors could not reduce the IL-17A expression level compared to control for both RA and PsA patients. These data indicate a different mechanism of action for TNFis compared to IL-17A and IL-6R inhibitors and suggest their inefficacy for testing biologics, such as IXE and TCZ, in the in vitro assay.

All in all, the inventors disclose a new in vitro assay for the prediction of response to each currently available TNFi (e.g., anti-TNF alpha antibody, mimetic receptor) in a personalized manner. The qRT-PCR technique was applied as a readout system for assessing differences in response to each TNFi using a gene expression which differentiated among the various responses to TNFi. The in vitro assay could be a successful approach to predict the prognosis and outcome of therapy with TNFis. It could be a clinically useful and cost-effective means of assisting rheumatologists in selecting among the available TNFis and in monitoring response to treatment over time.

Therefore, the present invention provides teachings and means so as to which subjects are suitable for TNFα inhibition therapy, and further which specific TNFα inhibitor should be prescribed for the suitable subject, thereby achieving an optimal therapeutic effect (e.g., personalized, and optimal medicine).

### EXAMPLE 9

### IL-17A mRNA and Protein are Differently Expressed in a Similar Manner Following Co-Culture with Different TNFi

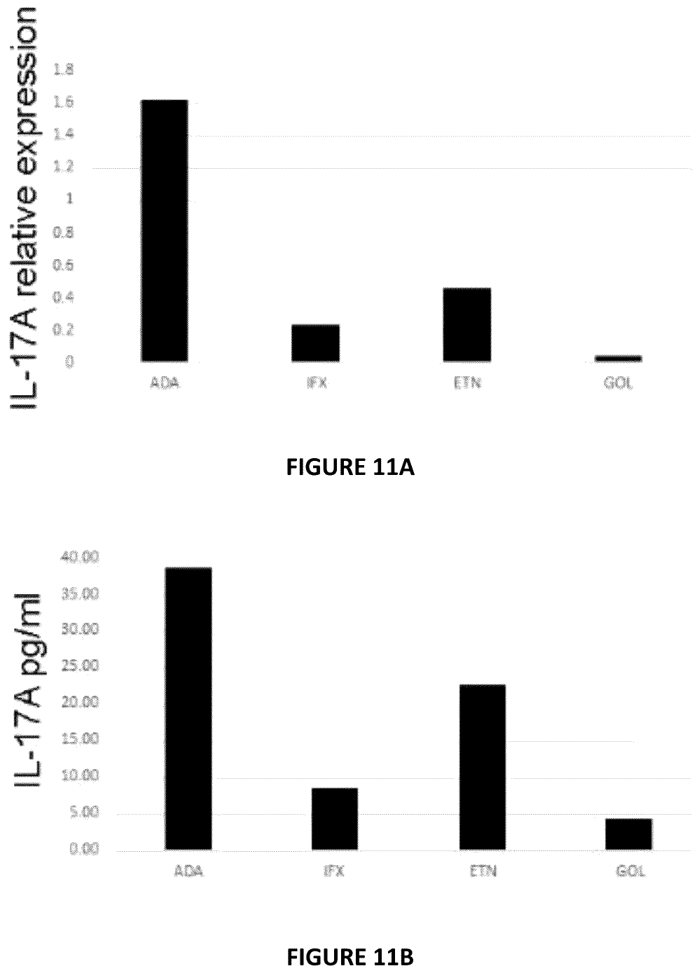

A comparison between RNA and protein expression levels of IL-17A in response to various TNFi was performed. The results show that a similar trend of IL-17A mRNA and protein expression levels were observed in supernatants (FIG. 11). IL-17A mRNA levels in PBMCs co-cultured with the different TNFi were found to be from the highest to lowest according to the following order: ADA-treated, ETN-treated, IFX-treated, and GOL-treated (FIG. 11A). Supernatants from the same samples exhibited a similar trend of IL-17A protein levels (FIG. 11B).

Therefore, the assay enables detecting changes in response to each TNFi as reflected by IL-17A expression both at the mRNA level (e.g., in cells) and protein level (e.g., in supernatants).

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

What is claimed is:

1. A method for treating a subject afflicted with a TNFα related disease, comprising the steps:
a. determining whether an expression level of IL-17 is reduced by at least 55% compared to a baseline, in a blood sample obtained or derived from a subject and contacted with at least one TNFα inhibitor for a period of at least 72 hours; and
b. administering to the subject determined as having at least 55% reduction in expression level of IL-17, a therapeutically effective amount of a TNFα inhibitor, thereby treating the subject afflicted with a TNFα related disease.

2. The method of claim 1, wherein said at least one TNFα inhibitor comprises a plurality of types of TNFα inhibitors.

3. The method of claim 2, wherein said blood sample of step (a) is independently contacted with said plurality of types of TNFα inhibitors.

4. The method of claim 2, further comprising a step of determining which of said plurality of types of TNFα inhibitors provides the greatest reduction in expression level of IL-17.

5. The method of claim 4, wherein said step (b) comprises administering to the subject the TNFα inhibitor determined as providing the greatest reduction in expression level of IL-17.

6. The method of claim 1, wherein said administering comprises intravenously administering or subcutaneously administering.

7. The method of claim 1, wherein said TNFα inhibitor is an anti-TNFα antibody or a TNFα mimicking receptor.

8. The method of claim 1, wherein said TNFα inhibitor is selected from the group consisting of: Infliximab, Adalimumab, Golimumab, Certolizumab pegol, and Etanercept.

9. The method of claim 1, wherein said subject is afflicted with a TNFα related disease being selected from the group consisting of: psoriatic arthritis, rheumatoid arthritis, ankylosing spondylitis, psoriasis, Crohn's disease, and ulcerative colitis.

10. The method of claim 1, wherein said sample comprises PBMCs derived form said subject.

11. The method of claim 1, wherein said contacting is for a period of at least 72 hours.

12. The method of claim 1, further comprising a step preceding step b, comprising extracting RNA from said contacted sample.

13. The method of claim 1, wherein said determining is by quantitative RT-PCR.

14. The method of claim 1, further comprising a step preceding step b, comprising extracting proteins from said contacted sample.

15. The method of claim 1, wherein said determining is by an immunoassay.

16. The method of claim 15, wherein said immunoassay is enzyme-linked immunosorbent assay (ELISA).
